(19) United States
(12) Patent Application Publication
(10) Pub. No.: US 2014/0358205 A1
(43) Pub. Date: Dec. 4, 2014

(54) METHOD AND APPARATUS OF EVAPORATIVE COOLING FOR AN ANIMAL

(71) Applicants: Mary Jo Margaret Robke, Louisville, KY (US); Vicki Shapero Lurie, Louisville, KY (US)

(72) Inventors: Mary Jo Margaret Robke, Louisville, KY (US); Vicki Shapero Lurie, Louisville, KY (US)

(21) Appl. No.: 14/288,023

(22) Filed: May 27, 2014

Related U.S. Application Data
(60) Provisional application No. 61/827,778, filed on May 28, 2013.
(60) Provisional application No. 61/827,782, filed on May 28, 2013.

(51) Int. Cl. A61D 1/00; A61F 7/10

(52) U.S. Cl. CPC A61D 1/00; A61F 7/10
USPC 607/109; 607/112; 607/108

(57) ABSTRACT

The present invention provides a method and apparatus of evaporative cooling which targets one or more of an animal's muscle tissue areas, as the head and neck, chest, shoulder, back or gluteal muscles, and leg and ankle of the body chest, shoulder, back and gluteal muscles, and does not otherwise restrict movement of the animal. The apparatus of the present invention is formed of a lower layer and an upper layer, which together form a pocket for receiving a cooling means, wherein the pocket is adapted to open and dose. The apparatus of the present invention is contoured to position around one or more body areas of the animal and secured in the position for evaporative cooling of the animal.

### CROSS-REFERENCE TO RELATED APPLICATION

The present invention claims priority to U.S. Provisional Application No. 61/827,778, filed May 28, 2013, entitled “COOLING BLANKET GEL DEVICE” (Robke and Lurie) and U.S. Provisional Application No. 61/827,782, filed May 28, 2013, entitled “COOLING GEL HEADPIECE DEVICE” (Robke and Lurie), which are incorporated herein in their entirety.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### FIELD OF THE INVENTION

The present invention relates generally to a method and apparatus for evaporative cooling for an animal. More particularly, the present invention pertains to an apparatus that is positioned around one or more areas of the body of an animal for evaporative cooling, allowing free movement of the animal.

### BACKGROUND OF THE INVENTION

Heat, humidity and activity induced exertion pose health risks for animals, including heat-stroke, stress, dehydration, lethargy and loss of appetite. Those who work closely with or interact with animals, such as owners, trainers, veterinarians, keepers or custodians, and others, often integrate numerous methods of cooling into an animal's daily routine. For example, known cooling methods for animals include water for consumption and external wash down, coat care such as grooming and sunscreen products, shade and ventilation, and electrolytes replenishing sodium loss during sweating.

The risk of heat, humidity and activity induced exertion is especially present in equine health, such as the equines at the show and competition circuit, farms and ranches. Equine animals include horses, as well as all members of the horse family. In equine care, in addition to the known cooling methods already identified, evaporative cooling is also a known option of cooling. Generally, evaporative is a combination the use of ice or ice packs for an application period, which may be instantaneous, such as pouring a bucket of ice over the equine, or for a longer term, such as positioning the ice or ice pack on a localized area, such as the spine, or legs for a defined period of time.

Such evaporative cooling for equines using ice or ice packs provide many uses and purposes. However, one drawback, which has heretofore been unrecognized in the prior art, is that the use of ice and ice packs often do not surround the muscle tissue of the head and neck, chest, shoulder and back, gluteal muscles, and leg and ankle of the body. Cooling the muscle tissue is effective for evaporative cooling of the equine. Additionally, the ice and ice packs are often difficult to position and result in slippage from initial positioning or restricted movement of the equine in consideration of the positioning. Furthermore, the equine are also less likely to accept any the ice or ice packs given the unpleasant sensation association with the initial positioning of the ice or ice pack upon the equine. Given these drawbacks, the use of ice or ice packs alone is not as effective at providing evaporative cooling of the equine.

So, an apparatus for evaporative cooling of the equine is a highly desirable method and apparatus. What is needed is a solution to address evaporative cooling of the equine by cooling muscle tissue that does not restrict movement of the animal.

The present invention overcomes the gaps of the prior art by providing a method and apparatus of evaporative cooling for an animal, where such apparatus does not substantially restrict movement of the animal. The present invention is useful in show and competition circuit, farms and ranches, veterinary and home use.

### SUMMARY OF THE INVENTION

The present invention provides a method and apparatus of evaporative cooling which targets one or more of the animal's muscle tissue of the head and neck, chest, shoulder, back and gluteal muscles, and leg and ankle of the body, and does not otherwise restrict movement of the animal. In one embodiment, the present invention is configured to equine use. In an alternate embodiment, the present invention is configured for canine and other animals. In one aspect, the present invention provides a contoured apparatus that substantially conforms to the shape of the head and neck of an animal, and the method for the same. In another aspect, the present invention provides an apparatus that substantially conforms to the shape to one or more of the chest, shoulder and back of an animal, and the method for the same. In another aspect, the present invention provides an apparatus that substantially conforms to the shape of the leg and ankle of an animal, and the method for the same. In another aspect, the present invention provides an apparatus that substantially conforms to the shape of one or more areas of the body of an animal, and the method for the same.

Furthermore given that the apparatus of the present invention substantially conforms to the contour of one or more areas of the animal body, the present invention also contemplates one or more securing means to minimize slippage of the apparatus and does not substantially restrict movement of the equine

The present invention is appropriate for many uses in a wide variety of settings, including equines at the show and competition circuit, farms and ranches, and any other site with an equine presence. Intended purposes and functions include promoting animal health and wellness, as well as other functions readily appreciated by one of ordinary skill in the art. In addition to its novel and nonobvious solutions to the problems mentioned above, the present invention also encompasses the recognition of those previously unidentified problems in the art.

### BRIEF DESCRIPTION OF DRAWINGS

In order to facilitate a full understanding of the present invention, reference is now made to the accompanying drawings, in which like elements are referenced with like numerals. These drawings should not be construed as limiting the present invention, but are intended to be exemplary and for reference.

FIG. 1 is a right side plan view of a cooling apparatus of an embodiment of the present invention.

FIG. 2 is a front plan view of a cooling apparatus of an embodiment of the present invention.

FIG. 3 is a plan view of the top surface of a cooling apparatus of an embodiment of the present invention.

FIG. 4 is a plan view of the bottom surface of a cooling apparatus of an embodiment of the present invention.

FIG. 5 is a right side plan view of a cooling apparatus of an embodiment of the present invention.

FIG. 6 is a left side plan view of a cooling apparatus of an embodiment of the present invention.

FIG. 7 is a plan view of the top surface of a cooling apparatus of an embodiment of the present invention.

FIG. 8 is a plan view of the bottom surface of a cooling apparatus of an embodiment of the present invention.

FIG. 9 is a right side plan view of a cooling apparatus of an embodiment of the present invention.

FIG. 10 is a right side plan view of a cooling apparatus of an embodiment of the present invention.

FIG. 11 is a plan view of the top surface of a cooling apparatus of an embodiment of the present invention.

FIG. 12 is a plan view of the bottom surface of a cooling apparatus of an embodiment of the present invention.

### DETAILED DESCRIPTION OF PREFERRED EMBODIMENT

The present invention will now be described in more detail with reference to exemplary embodiments as shown in the accompanying drawings. While the present invention is described herein with reference to the exemplary embodiments, it should be understood that the present invention is not limited to such exemplary embodiments. Those possessing ordinary skill in the art and having access to the teachings herein will recognize additional implementations, modifications, and embodiments, as well as other applications for use of the invention, which are fully contemplated herein as within the scope of the present invention as disclosed and claimed herein, and with respect to which the present invention could be of significant utility.

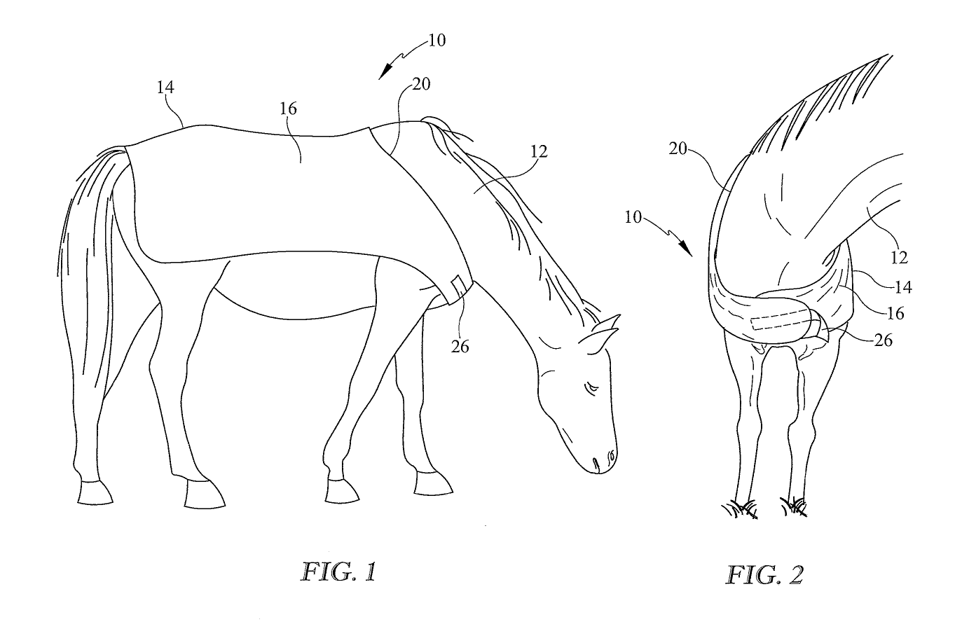

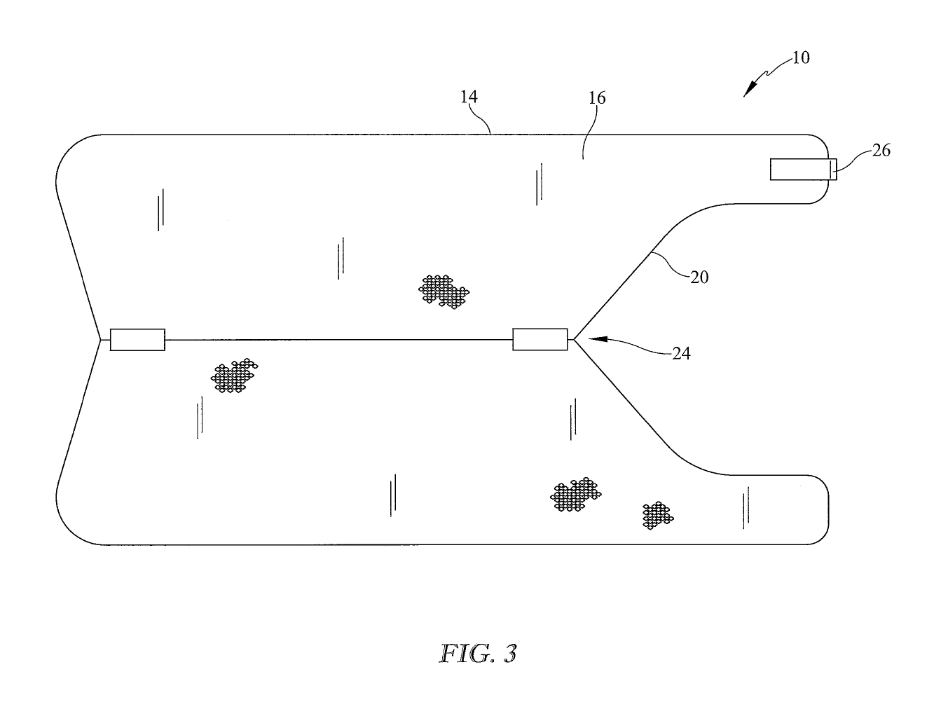

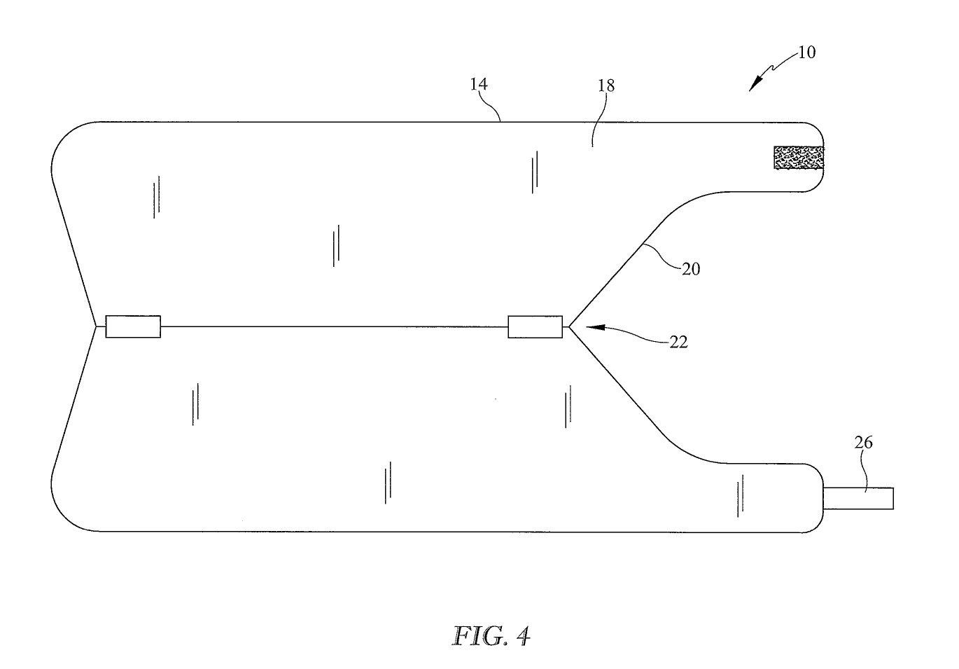

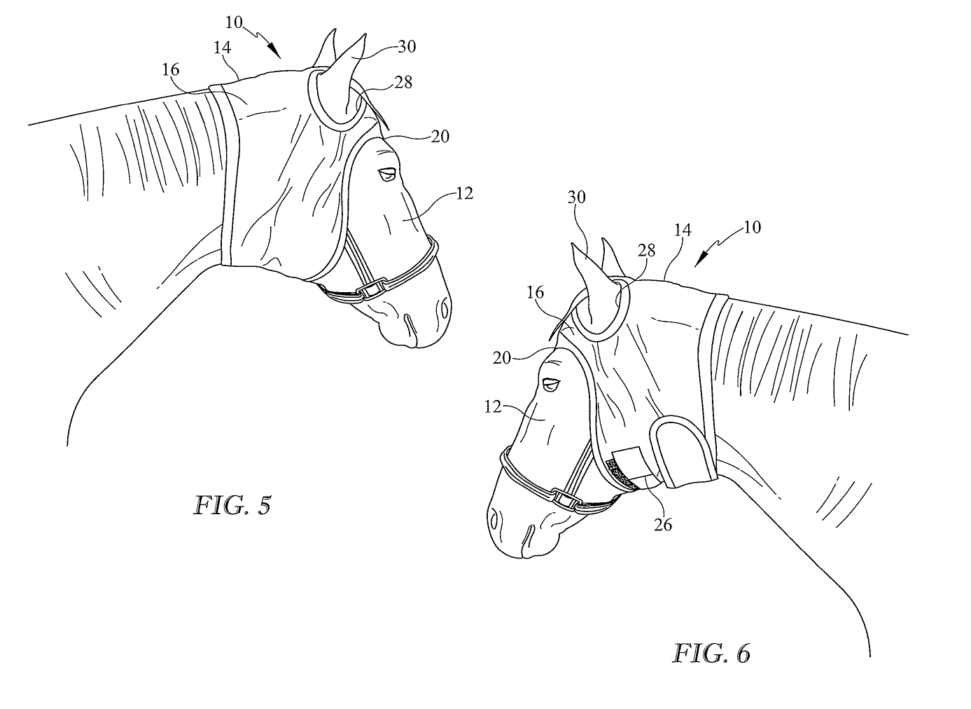

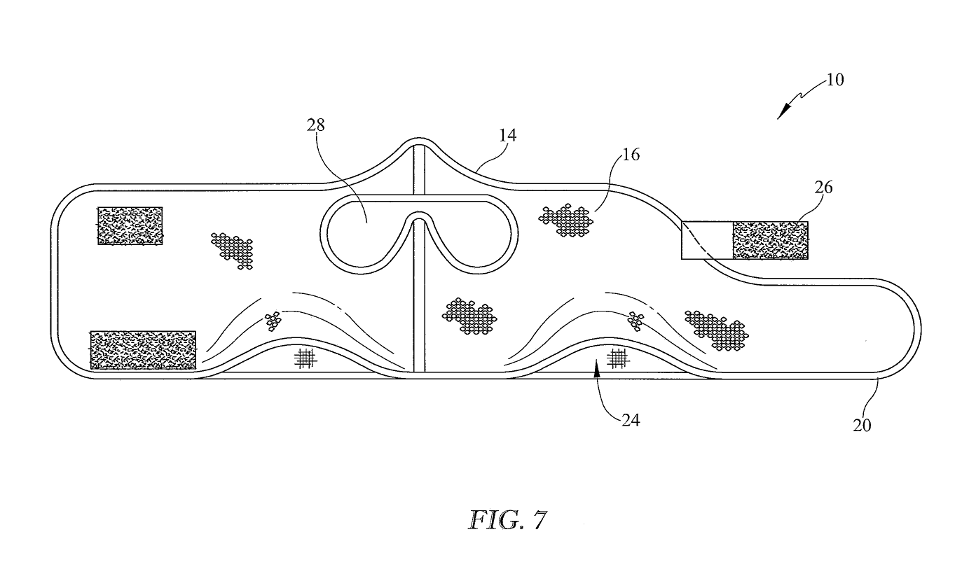

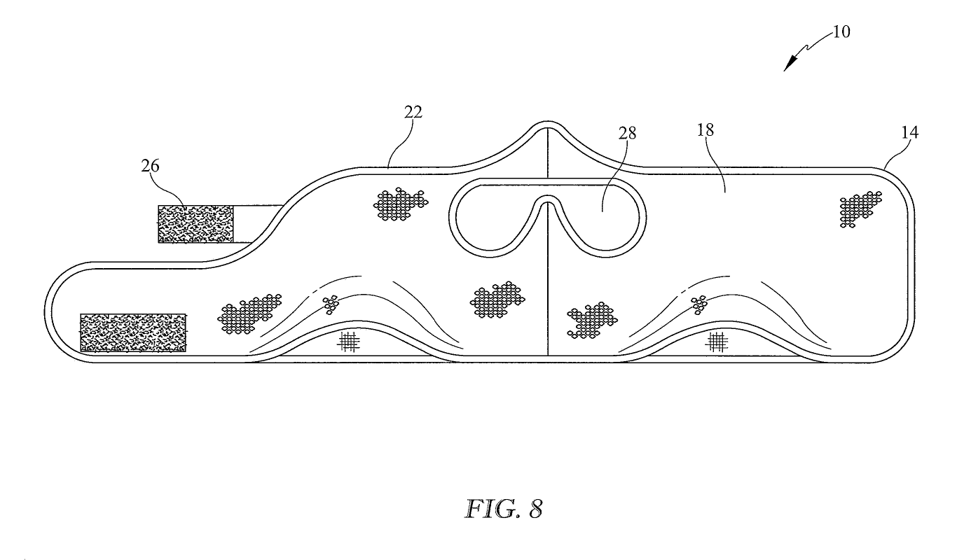

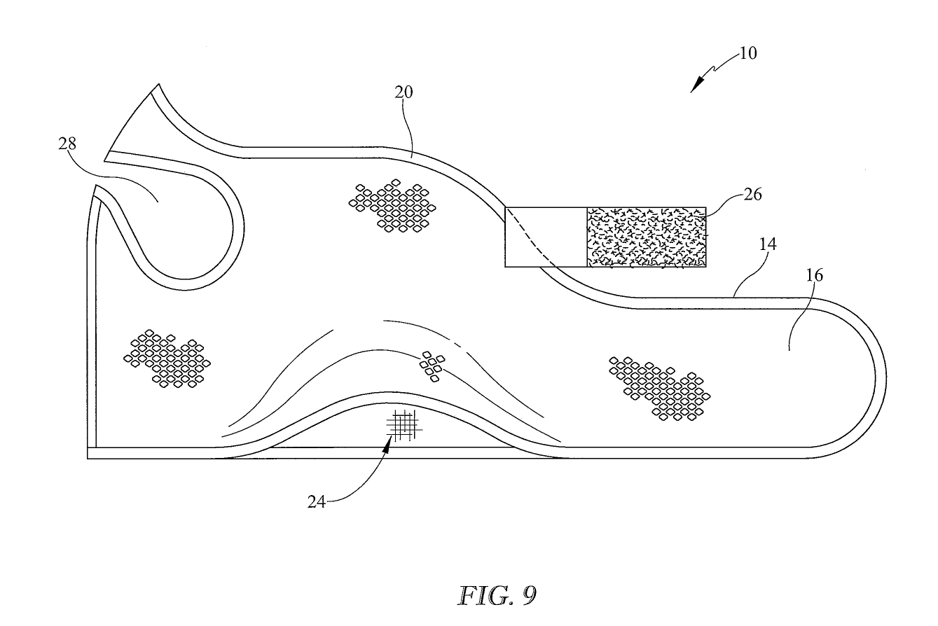

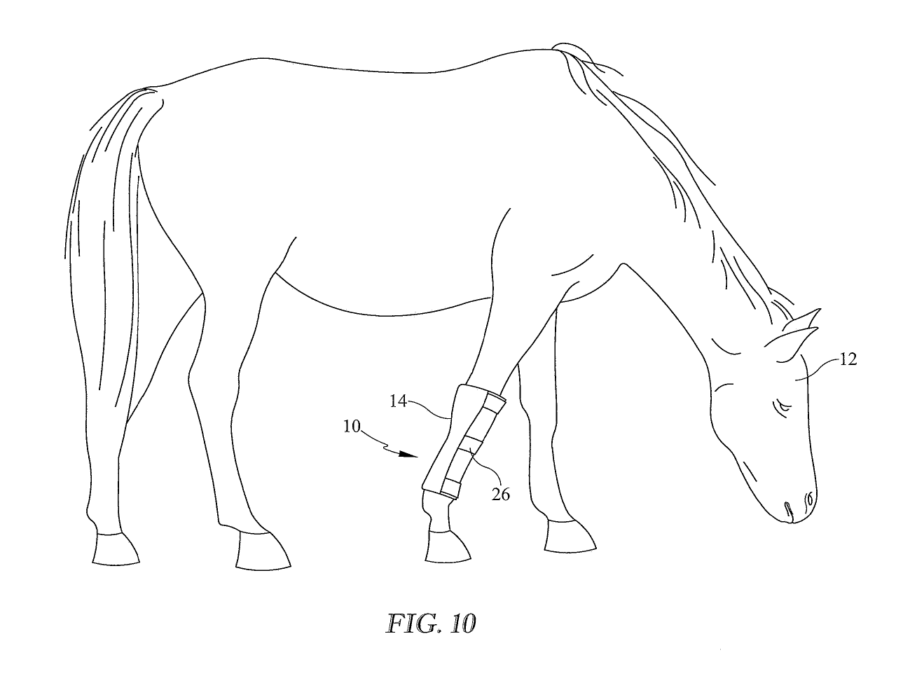

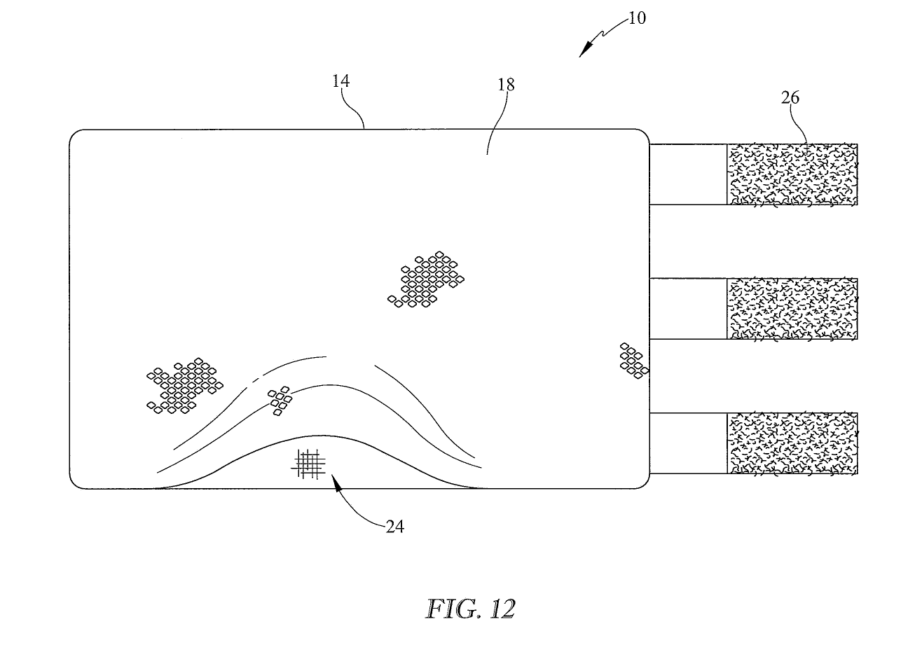

Referring to FIGS. 1-12, where like apparatus is identified with like reference numerals, apparatus 10 for evaporative cooling of an animal 12 according to the present invention. As is illustrated in FIGS. 1-12, the apparatus comprises a contoured pad 14 having an upper layer 16 and a lower layer 18. The upper layer 16 has an upper layer perimeter 20 of substantially the same dimension as that of the lower layer 18, having the lower layer perimeter 22. It is conceived that it may be desirable for the lower layer 18 to be a material that is permeable to temperature emitted from a cooling element positioned in between the upper layer 16 and the lower layer 18 toward the one or more areas of the animal body. In one embodiment, the lower layer 18 is a permeable, open cell durable mesh fabric. In an alternate embodiment, the lower layer 18 is a flexible, porous material. It is conceived that it may be desirable for the apparatus 10 to have properties in varying degrees of flexibility, water impermeability and aesthetic characteristics and it is conceived that the upper layer 16 shall have material construction to reflect such determined characteristic. In one embodiment, the upper layer 16 is a breathable fabric. In an alternate embodiment, the upper layer 16 is a flexible radiant barrier, as a reflective fabric material. The contoured pad 14 substantially conforms to the contour of portions of the body of the animal 12 selected from the group consisting of portions of the head and body, portions of the chest, shoulder and back, portions of the leg and ankle, portions of the gluteal muscles, and combinations thereof.

The upper layer 16 and the lower layer 14 have a set of pockets 24 formed between the upper layer 16 and the lower layer 14. The set of pockets 24 has one or more closing means adapted to open and close the set of pockets 24. The closing means is selected from the group consisting of a thread system, a hook and loop system, a button system, a male/female snap system, a buckle system, a clip system, a clasp system, a grommet system, a tie system, a zipper system, and combinations thereof. One or more cooling elements is removably positioned within the set of pockets 24. The cooling elements aid in the evaporative cooling of the animal. The cooling element is selected from the group consisting of a gel cooling pad, gel cooling sheet, ice pack, thermoelectric cooling element, refrigerant, and combinations thereof. The cooling element is separated from the animal 12 by the lower layer 14 and the cooling element permeates through the lower layer 14 for evaporative cooling, to pull heat from within the muscle tissue of the animal 12. The cooling of the animal 12 promotes health and wellness.

The contoured pad 14 is secured about the body of the animal 12 by a securing means 26. The cooling element within the contoured pad 14, as secured about the body of the animal 12, as targets one or more of the animal's 12 muscle tissue for heat reduction. The securing means 26 is adapted to secure the pad 14 around a selected body area of the animal 12. The securing means 26 is further adapted to provide for substantially unrestricted movement of the animal 12 for minimized slippage. The securing means 26 is selected from the group consisting of a hook and loop system, a button system, a male/female snap system, a buckle system, a clip system, a clasp system, a grommet system, a tie system, a zipper system, and combinations thereof.

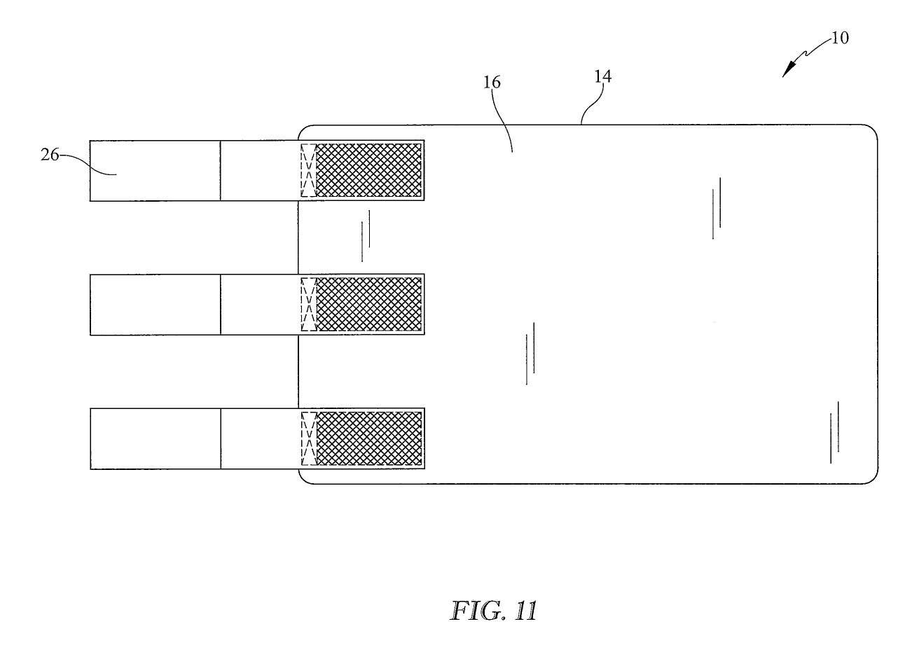

As is illustrated in FIGS. 1-4, in one embodiment of the apparatus 10, the contoured pad 14 substantially conforms to the contour of portions of the chest, shoulder and back and portions of the gluteal muscles of the animal 12. As is illustrated in FIGS. 5-9, in an alternate embodiment of the apparatus 10, the contoured pad 14 substantially conforms to the contour of portions of the head and body of the animal 12. As is illustrated in FIGS. 10-12, in another alternate embodiment of the apparatus 10, the contoured pad 14 substantially conforms to the contour of portions of the leg and ankle of the animal 12.

As is illustrated in FIGS. 5-9, the contoured pad 14 has at least one opening 28 adapted to allow at least one extremity, appendage or limb 30 of the animal 12 to extend through the contoured pad 14. In the embodiment of FIGS. 5-9, the extremity, appendage or limb 30 of the animal 12 is an ear.

In an alternate embodiment, the apparatus 10 further comprising a case to contain the apparatus 10. Additionally, in embodiments of the present invention, the animal 12 may be an equine, as a horse or member of the horse family, a canine, a human being or various other creatures.

The present invention is not to be limited in scope by the specific embodiments described herein. It is fully contemplated that other various embodiments of and modifications to the present invention, in addition to those described herein, will become apparent to those of ordinary skill in the art from the foregoing description and accompanying drawings. Thus, such other embodiments and modifications are intended to fall within the scope of the following appended claims. Further, although the present invention has been described herein in the context of particular embodiments and implementations and applications and in particular environments, those of ordinary skill in the art will appreciate that its usefulness is not limited thereto and that the present invention can be beneficially applied in any number of ways and environments for any number of purposes. Accordingly, the claims set forth below should be construed in view of the full breadth and spirit of the present invention as disclosed herein.

What is claimed is:

1. A method of evaporative cooling for an equine, the method comprising:
positioning a contoured apparatus around a body area of an equine, said apparatus having a permeable lower layer for contacting the equine's body area, an upper layer having an outer perimeter of substantially the same dimension as that of the lower layer, a set of pockets adapted to open and close, said pockets formed between the lower layer and upper layer, and the apparatus further comprising a securing means adapted to secure the apparatus around the equine body area;
inserting an inner core into at least one pocket of the apparatus, the inner core further comprising one or more cooling element adapted to aid in evaporative cooling of an equine;
closing the pocket of the apparatus to prevent the escape of the inner core; and
securing the apparatus around the body area of the equine, wherein said securing means is adapted to provide for substantially unrestricted movement of the equine.

2. A method in accordance with claim 1, further comprising a contoured apparatus conforming to the contour of portions of the equine body selected from the group consisting of portions of the head and body, portions of the chest, shoulder and back, portions of the leg and ankle, or portions of the gluteal muscles, and combinations thereof.

3. A method in accordance with claim 1, wherein the cooling element is selected from the group consisting of a gel cooling pad, gel cooling sheet, ice pack, thermoelectric cooling element or refrigerant, and combinations thereof.

4. A method in accordance with claim 1, further comprising a securing means is selected from the group consisting of a hook and loop system, a button system, a male/female snap system, a buckle system, a clip system, a clasp system, a grommet system, a tie system, a zipper system, and combinations thereof.

5. An apparatus for evaporative cooling of an equine, the apparatus comprising:
a contoured pad having an upper layer and a lower layer, an upper layer having an outer perimeter of substantially the same dimension as that of the lower layer;
a set of pockets formed between the upper layer and the lower layer, said pockets having one or more closing means adapted to open and close an opening to the pocket;
an inner core positioned in at least one pocket of the pad, said inner core comprising one or more cooling elements, said cooling elements adapted to aid in evaporative cooling of an equine; and
a securing means adapted to secure the pad around a body area of an equine, said securing means further adapted to provide for substantially unrestricted movement of the equine.

6. An apparatus in accordance with claim 5, wherein said contoured pad substantially conforms to the contour of portions of the equine body selected from the group consisting of portions of the head and body, portions of the chest, shoulder and back, portions of the leg and ankle, portions of the gluteal muscles, and combinations thereof.

7. An apparatus in accordance with claim 5, wherein said closing means is selected from the group consisting of a thread system, a hook and loop system, a button system, a male/female snap system, a buckle system, a clip system, a clasp system, a grommet system, a tie system, a zipper system, and combinations thereof.

8. An apparatus in accordance with claim 5, wherein said cooling element is selected from the group consisting of a gel cooling pad, gel cooling sheet, ice pack, thermoelectric cooling element, refrigerant, and combinations thereof.

9. An apparatus in accordance with claim 5, wherein said securing means is selected from the group consisting of a hook and loop system, a button system, a male/female snap system, a buckle system, a clip system, a clasp system, a grommet system, a tie system, a zipper system, and combinations thereof.

10. An apparatus in accordance with claim 5, further comprising at least one opening adapted to allow at least one extremity, appendage or limb of the equine to extend through the contoured pad.

11. An apparatus in accordance with claim 5, wherein said lower layer is a material that is permeable to temperature emitted from the cooling element toward the one or more areas of the equine.

12. An apparatus in accordance with claim 5, further comprising a case to contain the apparatus.

13. An apparatus for evaporative cooling of an animal, the apparatus comprising:
a first layer of permeable material having an outer perimeter;
a second layer of material having an outer perimeter of substantially the same dimension as that of the first layer;
the first layer and second layer being attached at their perimeters in a manner that forms a set of pockets between the first layer and the second layer, and that provides an opening for access into at least one pocket;
a removable insert as one or more cooling elements positioned within at least one pocket, said cooling elements adapted to aid in evaporative cooling of an animal;
said pocket further comprising one or more closing means adapted to open and close the opening to the pocket, said closing means adapted to allow removal of the insert and to prevent the unintended escape of the removable insert; and
a securing means to substantially secure the pocket around one or more areas of the animal body, wherein said securing means is adapted to provide for substantially unrestricted movement of the animal.

14. An apparatus in accordance with claim 13, wherein said perimeter of first layer and second later substantially conforms to the contour of portions of the animal body selected from the group consisting of portions of the head and body, portions of the chest, shoulder and back, portions of the leg and ankle, portions of the gluteal muscles, and combinations thereof.

15. An apparatus in accordance with claim 13, wherein said closing means is selected from the group consisting of a thread system, a hook and loop system, a button system, a male/female snap system, a buckle system, a clip system, a clasp system, a grommet system, a tie system, a zipper system, and combinations thereof.

16. An apparatus in accordance with claim 13, wherein said cooling element is selected from the group consisting of a gel cooling pad, gel cooling sheet, ice pack, thermoelectric cooling element, refrigerant, and combinations thereof.

17. An apparatus in accordance with claim 13, wherein said securing means is selected from the group consisting of a hook and loop system, a button system, a male/female snap system, a buckle system, a clip system, a clasp system, a grommet system, a tie system, a zipper system, and combinations thereof.

18. An apparatus in accordance with claim 13, further comprising at least one opening adapted to allow at least one extremity, appendage or limb of the animal to extend through the contoured pad.

19. An apparatus in accordance with claim 13, wherein said first layer is a material that is permeable to temperature emitted from the cooling element toward the one or more areas of the animal body.

20. An apparatus in accordance with claim 13, further comprising a case to contain the apparatus.
